(19) United States
(12) Patent Application Publication
(10) Pub. No.: US 2012/0059321 A1
(43) Pub. Date: Mar. 8, 2012

(54) TUNNELER FOR BI-DIRECTIONAL TUNNELING

(75) Inventors: Alisha M. Hammond, West Jordan, UT (US); Matthew W. Bown, West Bountiful, UT (US); Jeremy C. Horton, Salt Lake City, UT (US); Spencer R. Thomas, Salt Lake City, UT (US)

(73) Assignee: C. R. BARD, INC., Murray Hill, NJ (US)

(21) Appl. No.: 13/221,630

(22) Filed: Aug. 30, 2011

Related U.S. Application Data
(60) Provisional application No. 61/378,266, filed on Aug. 30, 2010.

(51) Int. Cl. A61B 17/34

(52) U.S. Cl. 604/164.05; 604/164.01; 606/108

(57) ABSTRACT

A tunneler for producing a subcutaneous tunnel in connection with the placement of a catheter within a body of a patient is disclosed. In one embodiment, the tunneler comprises an elongate tunneler member and a sheath. The tunneler member includes a handle on its proximal end and a tapered distal end. The sheath defines an elongate lumen inside which the tunneler member is initially received. The tunneler member is configured to define a tunnel through subcutaneous tissue while disposed in the sheath, then is removable from the sheath lumen thereafter. The sheath is configured to remain within the tunnel so that a catheter can be inserted through the sheath within the tunnel. The sheath is splittable in one embodiment so as to enable its removal from the tunnel while leaving the catheter in place.

### CROSS-REFERENCE TO RELATED APPLICATIONS

This application claims the benefit of U.S. Provisional Patent Application No. 61/378,266, filed Aug. 30, 2010, and entitled “Tunneler for Forward and Reverse Tunneling,” which is incorporated herein by reference in its entirety.

### BRIEF SUMMARY

Briefly summarized, embodiments of the present invention are directed to a tunneler for producing a subcutaneous tunnel in connection with the placement of a catheter within a body of a patient. In one embodiment, the tunneler comprises an elongate tunneler member and a sheath. The tunneler member includes a handle on its proximal end and a tapered distal end. The sheath defines an elongate lumen inside which the tunneler member is initially received. The tunneler member is configured to define a tunnel through subcutaneous tissue while disposed in the sheath, then is removable from the sheath lumen thereafter. The sheath is configured to remain within the tunnel so that a catheter can be inserted through the sheath within the tunnel. The sheath is splittable in one embodiment so as to enable its removal from the tunnel while leaving the catheter in place.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIGS. 1A-1C show various views of a tunneler configured in accordance with one embodiment;

FIG. 2 shows another tunneler in accordance with one embodiment;

FIG. 3 shows a cross sectional view of a tunneler in accordance with one embodiment;

FIGS. 4A and 4B show various views of a tunneler in accordance with one embodiment;

FIG. 5 is a cross sectional view of a tunneler in accordance with one embodiment;

FIGS. 6A and 6B show various views of a tunneler system configured in accordance with one embodiment; and

FIG. 7 is a cross sectional view of a tunneler in accordance with one embodiment.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the present invention, and are neither limiting nor necessarily drawn to scale.

For clarity it is to be understood that the word “proximal” refers to a direction relatively closer to a clinician using the device to be described herein, while the word “distal” refers to a direction relatively further from the clinician. For example, the end of a catheter placed within the body of a patient is considered a distal end of the catheter, while the catheter end remaining outside the body is a proximal end of the catheter. Also, the words “including,” “has,” and “having,” as used herein, including the claims, shall have the same meaning as the word “comprising.”

Embodiments of the present invention are generally directed to a tunneler for producing subcutaneous tunnels in connection with the placement of a catheter within a body of a patient. In one embodiment, the catheter is attached at one end to an access port that is also subcutaneously implanted in a pocket under the patient's skin. The other end of the catheter extends through the tunnel produced by the tunneler between the pocket and a vessel entry point where the catheter enters into the patient's vasculature. One possible vessel entry point for catheter entry into the vasculature includes the internal jugular vein, for instance. The tunneler embodiments to be described herein are bi-directional so as to enable the tunnel to be created in either a forward direction (i.e., from the port pocket to the vessel entry point) or reverse direction (i.e., from the vessel entry point to the port pocket). Further, the tunnelers to be described herein in connection with selected embodiments enable tunneling for catheters having both open and closed (valved) distal ends.

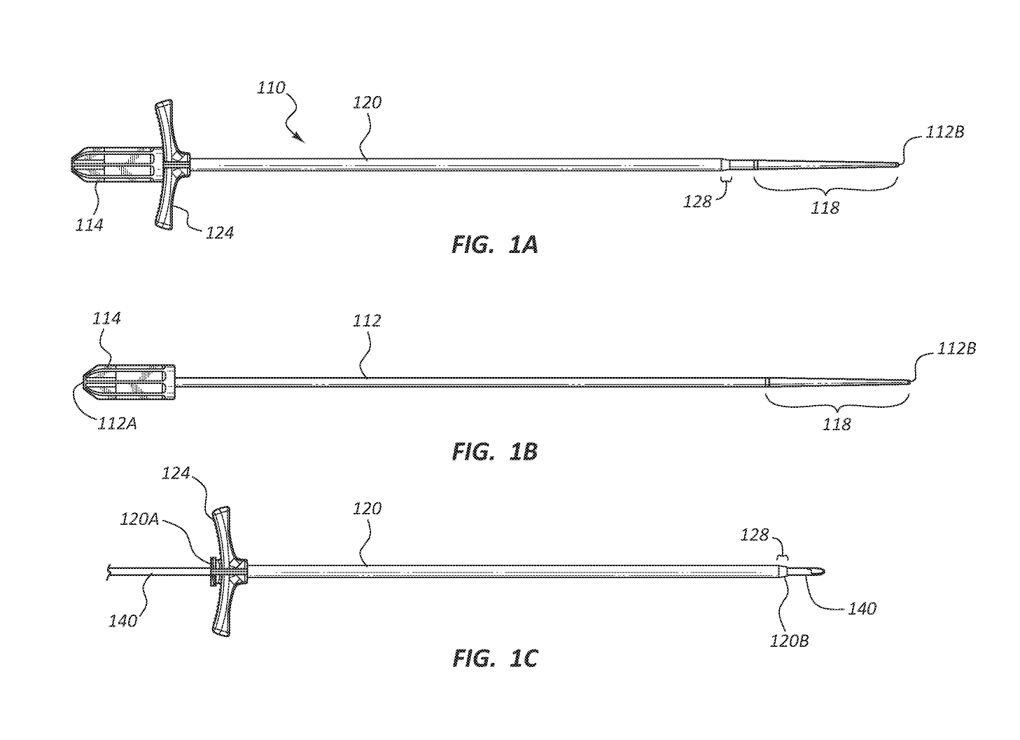

FIGS. 1A-1C show a tunneler 110 configured according to one embodiment, generally including a tunneler member 112 and a sheath 120. As best seen in FIG. 1B, the tunneler member 112 includes at its proximal end 112A a handle 114 for grasping the member during use. The body of the tunneler member 112 extends distally from the handle 114 and is substantially cylindrical, with the exception of a distal portion 118, which tapers down to a distal end 112B of the member so as to ease passage thereof through the subcutaneous tissue of a patient during a tunneling procedure. Such tunneling procedures are common, for instance, in providing a pathway for a catheter to extend from an access port positioned in a subcutaneous pocket and an entry site for insertion of the catheter into the vessel, such as the internal jugular vein, for instance.

The tunneler member 112 is removably received within the sheath 120. As best seen in FIG. 1C, the sheath 120 includes a handle 124 at the proximal end 120A thereof and a hollow, substantially cylindrical body extending distally from the handle. A relatively short distal portion 128 of the sheath 120 tapers down in outer diameter to the distal end 120B thereof so as to provide a smooth transition between the sheath distal portion and the adjacent outer surface of the tunneler member 112 when the tunneler member is disposed within the sheath.

FIG. 1A shows the tunneler 110 ready for a tunneling procedure, wherein the tunneler member 112 is disposed within the sheath 120. In the present embodiment, the tunneler handle 114 and the sheath handle 124 are threadably engageable with one another so as to maintain the configuration shown in FIG. 1A, but it is appreciated that other schemes can be employed to mate the tunneler member and sheath together.

Configured as shown in FIG. 1A, the tunneler 110 is employed in one embodiment to define a subcutaneous tunnel beneath the patient's skin, such as between a subcutaneous pocket in which an access port is disposed and a vessel entry point into a vein or other vessel of the patient. Once the tunneler 110 has been employed by a clinician to define the subcutaneous tunnel, the tunneler member 112 is removed from the sheath 120, which remains in place within the tunnel. As mentioned, removal of the tunneler member 112 from the sheath 120 in the present embodiment involves threadably disengaging the tunneler member handle 114 from the sheath handle 124.

After removal of the tunneler member 112, the hollow sheath 120 remains in place within the tunnel, thus providing a conduit through which a catheter 140 can be inserted in either a forward (e.g., toward the vessel entry site) or reverse (e.g., toward the pocket site) direction. Disposal of the catheter 140 within the lumen of the sheath 120 is shown in FIG. 1C.

In the present embodiment, the handle 124 and body of the sheath 120 are splittable into two or more pieces such that, when the catheter 140 has been placed as desired, the sheath can be peeled away from about the catheter and removed from the tunnel, thus leaving only the catheter in place within the tunnel. It is appreciated that the sheath can be manufactured from suitable materials, such as PTFE FEP, plastics, rubber, etc., to enable it to be split. The tunneler member can include stainless steel, titanium, other medical grade metals, rigid plastics, or other suitable materials.

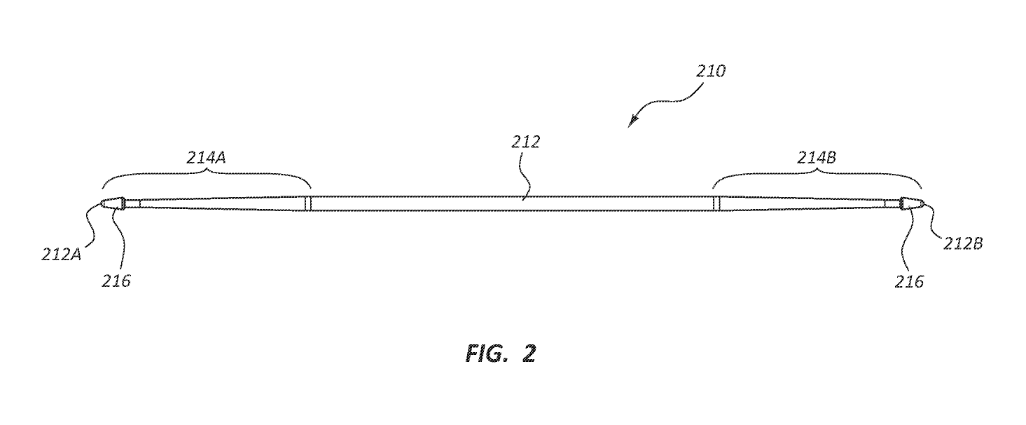

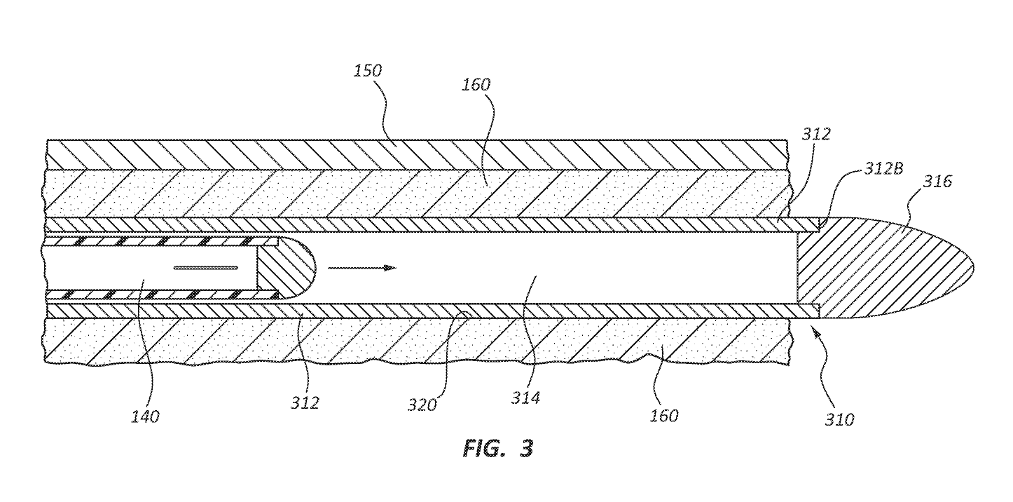

FIG. 2 depicts a tunneler 210 according to another embodiment, including a dual-ended body 212 defining proximal body portion 214A tapering down to a proximal end 212A and a distal body portion 214B tapering down to a distal end 212B. Barbs 216 are included at either end 212A, 212B of the tunneler 210 and are configured to both enable frictional engagement with a catheter placed on either end and to tunnel through tissue. So configured, the tunneler 210 can receive a catheter on one of either end 212A, 212B of the tunneler body 212 and advance through the tissue led by the other end of the tunneler body. This configuration enables the catheter to be pulled by the tunneler through a tunnel in either a forward or reverse direction regardless of which end the catheter is attached to.

FIG. 3 shows a tunneler 310 in accordance with one embodiment, wherein the tunneler includes an elongate hollow body 312 defining a lumen 314 that extends between a proximal end and a distal end 312B. The tunneler 310 further includes a tip 316 that is removably attached to the distal end 312B of the tunneler body 312 such as via threadable engagement, friction fit, snap fit, etc.

So configured, the tunneler 310 can be employed to define a subcutaneous tunnel in either the forward or reverse direction and to subsequently provide a conduit through which a catheter can be advanced through the tunnel. Particularly, once it has been used by a clinician to define a tunnel 320 (as shown in FIG. 3), the tunneler 310 is kept in place within the tunnel. The tunneler tip 316 is then removed to provide, together with the open proximal end of the tunneler, access to the tunneler lumen 314 as an open conduit through which the closed end or other type of catheter 140 can be inserted in either the forward or reverse direction to extend between the port pocket and the vessel insertion site, or other path. Once the catheter is placed, the tunneler body can be slid out of the tunnel and off the catheter. Note here that this and the other tunnelers herein can be configured as single-use or reusable components.

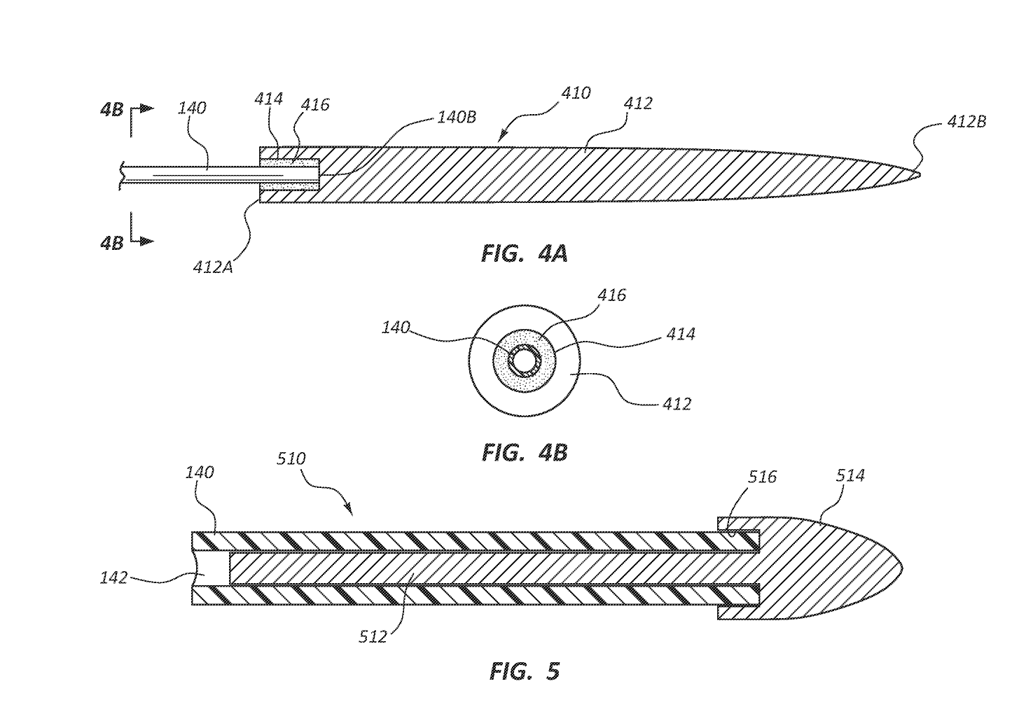

FIGS. 4A and 4B show a tunneler 410 in accordance with one embodiment, which includes an elongate body 412 defining a proximal end 412A and a distal end 412B to which the body tapers down for use in defining the tunnel. A cylindrical cavity 414 is defined in the proximal end 412A of the tunneler body 412. A hollow sleeve 416 including a compliant material such as silicone or other suitable material is disposed within the cavity of the second end 412B.

So disposed, the sleeve 416 is operative to grip and retain a distal portion of the catheter 140 when the distal end 140B thereof is inserted into the cavity 414 and into engagement with the sleeve. The sleeve 416 in one embodiment is sized with an inside diameter slightly smaller than an outer diameter of the catheter 140 to establish a compressive friction fit with a distal portion of the catheter, thus enabling the catheter to follow the tunneler 410 as the body 412 thereof is advanced by the clinician in forming the subcutaneous tunnel. Once the catheter 140 is in place within the tunnel, the distal portion thereof can be removed from the sleeve 414 by a sufficient pulling force. Note that the tunneler configuration of FIGS. 4A and 4B enables both open and close-ended catheters to be used with the present tunneler 410. In the present embodiment, the sleeve 416 defines a hollow cylinder; in another embodiment, the sleeve can include a plurality of gripping fingers that extend inwardly to grasp the catheter. The sleeve can also define a barb structure for engaging the catheter. In addition to silicone, other suitable materials can be employed to manufacture the sleeve, such as materials that include a tacky surface for instance. These and other variations are therefore contemplated.

FIG. 5 shows a tunneler 510 in accordance with one embodiment, which includes an elongate body 512 terminating in a distal tip 514. The portion of the tip 514 adjacent the elongate portion of the body 512 defines an annular cavity 516. Before use, the tunneler 510 is joined with the catheter 140 such that the elongate tunneler body 512 is received into a lumen 142 of the catheter 140 and an end of the catheter is received into the annular cavity 516 defined by the tunneler tip 514. Thus, as seen the tunneler tip 514 defines an outer diameter that is greater relative to the outer diameter of the catheter.

So configured, the tunneler 510 enables the catheter 140 to be advanced as the clinician uses the tunneler to define the subcutaneous tunnel. Once the catheter 140 is in final position within the tunnel, the tunneler 510 is removed from the catheter and the tunnel. Note that in one embodiment the tunneler tip is removable from the elongate body so as to facilitate ease of tunneler removal.

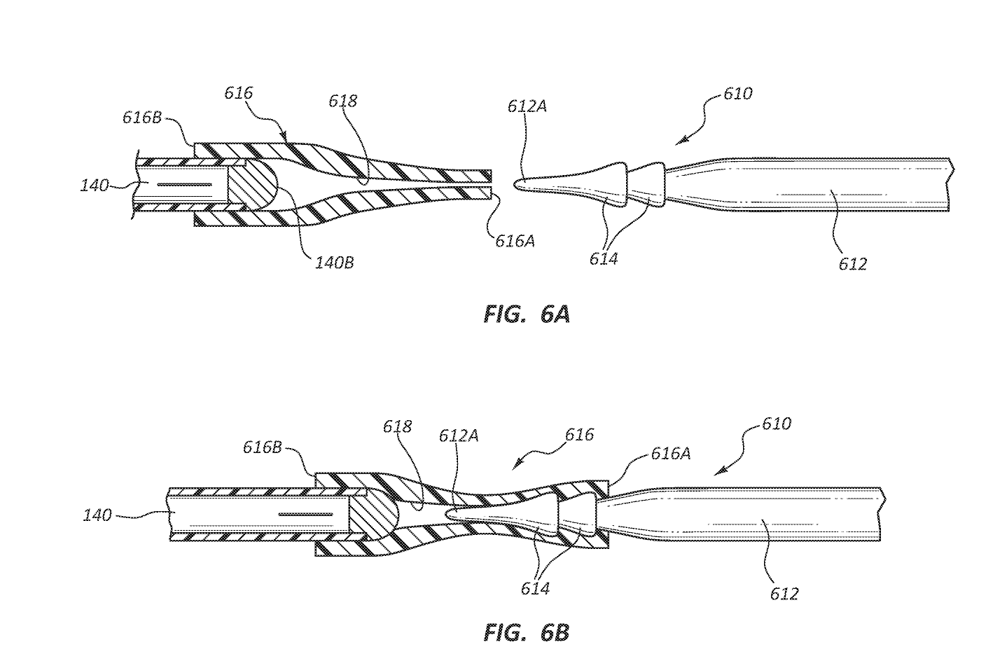

FIGS. 6A and 6B show various details of a tunneler system 610 according to one embodiment. The system 610 includes an elongate tunneler member 612 including one or more barbs 614 at its proximal end 612A. A compliant adapter 616 is also included with the system 610, defining a lumen 618 that extends between open first end 616A and open second end 616B. The adapter 616 is configured to receive through the first end 616A the proximal portion of the tunneler 612 including the barbs 614 (FIG. 6B) and to receive through the second end 616B a distal end 140B of the catheter 140, such as the close ended catheter shown in FIGS. 6A and 6B, or an open ended catheter. The compliant nature of the material included in the adapter 616 enables the adapter to frictionally engage the tunneler member 612 and the catheter 140, thus enabling the catheter to be advanced through the tunnel formed by the tunneler member. Application of sufficient separation force to the adapter 616 and one of the tunneler member 612 and catheter 140 enables the components to be separated when tunneling is complete. In one embodiment, the adapter 616 includes silicone, though other suitable compliant materials may also be used as appreciated by one skilled in the art. Note that in one embodiment the inner surface of the adapter can include engagement features, such as compliant, annular barbs for example, to assist in maintaining engagement with the tunneler member, the catheter, or both.

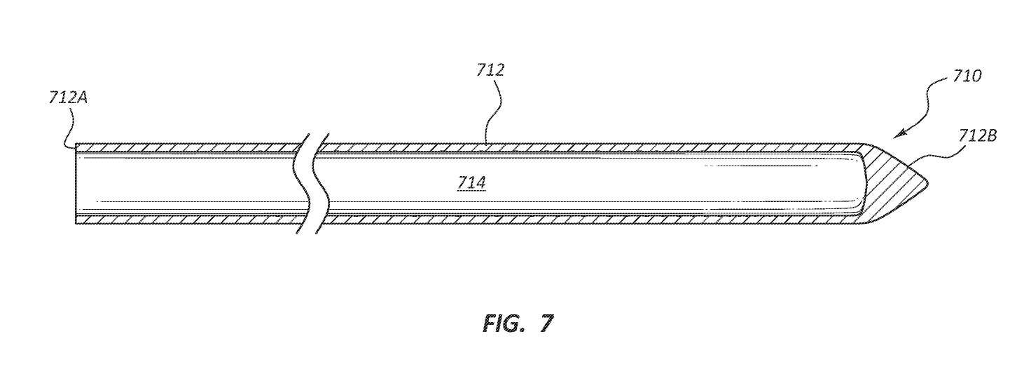

FIG. 7 shows details of a tunneler 710 according to one embodiment, wherein the tunneler includes an elongate body 712 defining a lumen 714 extending between an open proximal end 712A and a closed distal end 712B. The distal end 712B defines a shaped tip so as to enable tunneling through subcutaneous tissue. The tunneler 710 is configured to receive within its lumen 714 a catheter, which resides within the tunneler while the tunneler is advanced by a clinician through subcutaneous tissue to define the tunnel. Once the tunnel has been formed, the tunneler 710 can be removed while the catheter remains in place in the tunnel. Closed and open ended catheters may be used with the tunneler 710.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. A tunneler, comprising:
an elongate tunneler member including a handle on a proximal end and a tapered distal end; and
a sheath member defining an elongate lumen inside which the tunneler member is received, the tunneler member configured to define a tunnel through subcutaneous tissue, the tunneler member being removable from the sheath lumen after defining the tunnel, the sheath configured to remain within the tunnel so that a catheter can be inserted through the sheath within the tunnel.

2. The tunneler as defined in claim 1, wherein the sheath is splittable to enable removal of the sheath from around the catheter within the tunnel while the catheter remains in place within the tunnel.

3. The tunneler as defined in claim 1, wherein a distal portion of the sheath is shaped to provide a substantially smooth transition between the distal portion of the sheath and an adjacent portion of the tunneler member.

4. The tunneler as defined in claim 1, wherein the sheath is sized to extend within the tunnel between a subcutaneous pocket for a medical device and a vessel entry site.

5. The tunneler as defined in claim 4, wherein the tunneler member is configured to define the tunnel in one of a direction toward the subcutaneous pocket and a direction toward the vessel entry site.

6. A tunneler, comprising:
a hollow elongate tunneler body defining a lumen extending between open proximal and distal ends; and
a tip covering the distal end of the tunneler body and shaped to assist in defining a subcutaneous tunnel, wherein the tip is removable from the tunneler body after defining the tunnel so as to enable a catheter to pass through the lumen of the tunneler body.

7. The tunneler as defined in claim 6, wherein the tip is tapered and wherein the tunneler body enables the catheter to extend between a port pocket placement site and a vessel entry site.

8. The tunneler as defined in claim 6, wherein the tunneler is substantially cylindrical and wherein the removable tip engages the tunneler body via at least one of a threadable engagement and a friction fit.

9. The tunneler as defined in claim 6, wherein the tunneler can be used to define the tunnel in at least one of a direction toward and a direction away from a vessel entry site.

10. A tunneler, comprising:
an elongate tunneler body including a tapered distal end;
a cavity defined on a proximal end of the tunneler body; and
an engagement member disposed in the cavity for frictionally engaging an end portion of a catheter inserted into the cavity so as to enable the tunneler to pull the catheter through a subcutaneous tunnel defined by the tunneler.

11. The tunneler as defined in claim 10, wherein the engagement member includes a sleeve disposed in the cavity, the sleeve compressively engaging an outer surface of the catheter.

12. The tunneler as defined in claim 11, wherein the sleeve includes silicone, and wherein the catheter is removably received into the sleeve.

13. The tunneler as defined in claim 12, wherein the sleeve is cylindrically shaped.

14. The tunneler as defined in claim 13, wherein an inner diameter of the sleeve is sized smaller relative an outer diameter of the catheter, and wherein the catheter is a closed end catheter.

15. A tunneler, comprising:
an elongate tunneler body sized to be received within a lumen of a catheter; and
a tip disposed at a distal end of the tunneler body, the tip remaining external to the catheter lumen and sized to define an outer diameter that is greater relative the outer diameter of the catheter.

16. The tunneler as defined in claim 15, wherein the tip defines an annular cavity about a junction of the tip with the tunneler body, and wherein the distal end of the catheter is received into the annular cavity.

17. The tunneler as defined in claim 16, wherein the annular cavity is sized to retain the distal end of the catheter in a friction fit.

18. The tunneler as defined in claim 16, wherein the annular cavity includes a sleeve for retaining the catheter distal end therein.

19. A tunneler system, comprising:
a tunneler including at least one barb on an end thereof; and
a compliant adapter member defining a lumen extending between first and second ends, wherein the adapter first end receives and frictionally engages the end of the tunneler including the at least one barb, and wherein the adapter second end receives and frictionally engages an end of a catheter so as to enable the tunneler to pull the catheter through a subcutaneous tunnel defined by the tunneler.

20. The tunneler as defined in claim 19, wherein the catheter is a closed end catheter.

21. The tunneler as defined in claim 19, wherein the adapter includes silicone and is substantially cylindrical at rest.

22. A method for inserting a catheter into a subcutaneous tunnel, the method comprising:
creating the tunnel by a tunneler, the tunneler including a tunneler member removably received within a sheath;
removing the tunneler member from the sheath, the sheath remaining in the tunnel;
inserting the catheter through the sheath so as to be disposed in the tunnel; and
removing the sheath.

23. The method for inserting as defined in claim 22, wherein removing the sheath further includes:
splitting the sheath apart so as to remove the sheath from about the catheter and from the tunnel, the catheter remaining in the tunnel.
